(19) United States
(12) Patent Application Publication
(10) Pub. No.: US 2013/0158437 A1
(43) Pub. Date: Jun. 20, 2013

(54) System For Diagnosing and Identifying Genetic Features, Genetic and Congenital Syndromes And Developmental Abnormalities

(71) Applicant: Sharon Moalem, New York, NY (US)

(72) Inventor: Sharon Moalem, New York, NY (US)

(21) Appl. No.: 13/714,954

(22) Filed: Dec. 14, 2012

Related U.S. Application Data
(60) Provisional application No. 61/570,732, filed on Dec. 14, 2011.

(51) Int. Cl. A61B 5/107; A61B 8/13; A61B 8/08; A61B 6/03; A61B 6/00; A61B 5/00; A61B 5/055

(52) U.S. Cl. CPC A61B 5/107; A61B 5/7246; A61B 5/7282; A61B 5/74; A61B 8/13; A61B 5/0075; A61B 5/0077; A61B 5/055; A61B 6/03; A61B 5/0035; A61B 6/5247; A61B 8/5261; A61B 5/7225
USPC 600/587

(57) ABSTRACT

The invention is a system for diagnosing and identifying of genetic characteristics, genetic syndromes, congenital syndromes, developmental and exposure related syndromes, and veterinary breed characteristics in fetuses, children and adults, in humans and animals. The system takes a set of facial anatomical measurements of an individual target and biological relatives of the individual target, normalizes the facial anatomical measurements, and compares the normalized measurements with a database of set of anatomical features associated with a desired diagnostic data set.

### FIELD OF INVENTION

This invention is a system for diagnosing and identifying genetic features, genetic and congenital syndromes and developmental abnormalities in fetuses, children and adults, in humans and animals.

### BACKGROUND

Many medical and genetic conditions and/or syndromes have specific physical features, such as increased interpupillary distance and abnormal skull shape, that are used by physicians or medical allied health workers as an aid in diagnoses. For example, increased distance between the eyes of an individual can be an indicator of a condition called Noonan or Waardenburg syndrome. But it is also found in more than thirty other conditions. To differentiate among such syndromes or conditions it helps to increase the amount of data one acquires, such as the shape of the hands and fingers. For example, hypertelorism with tapering fingers and bulbous nose are features that are more likely to be consistent with Coffin-Lowry Syndrome. Currently, there are many drawbacks of using this type of technique to try to evaluate for disease. Firstly, many of the measurements are difficult and time-consuming to obtain, since most children are not cooperative enough to allow for all the measurements to be properly acquired manually. Even if all the measurements are acquired, an operator currently still needs to manually mark and measure these features even when a still image is used, which is highly time-consuming and would not be feasible for use as a screening tool for millions of children or adults. As well, currently many diagnoses are based on visual interpretations combining many features (or gestalt) of facial and/or other physical features that are highly subjective in nature and lack reproducibility and are highly operator dependent. Many of these variances are universal across ethnicities, but most health care practitioners lack the training to recognize them. Failing to recognize specific physical traits can often lead to inappropriate genetic testing and unnecessary costs totaling thousands of dollars.

Where a physician would usually have to request a wide array of tests for each genetic disorder, costing upwards of a few thousand dollars per test, this invention will be able to provide a rationale for testing that will reduce the financial burden on the individual, their families and on medical systems.

There are also many syndromes which do not have a genetic basis such as Fetal Alcohol Spectrum Disorders (FASD) that are diagnosed retrospective to a fetal alcohol exposure is largely based on facial features and cognitive issues seen after birth, such as a smooth philtrum, flat nasal bridge, thin upper lip and epicanthal folds, shortened palpebral fissures. But many of these physical features seen after birth are also visible during fetal life, but identification can be limited by the same constraints currently facing healthcare operators for genetic syndromes, which is lack of experience in identifying key markers. This invention overcomes these limitations by assessing the fetus for the pattern of minor fetal anomalies including intrauterine growth restriction seen in children exposed to alcohol.

Essentially, this invention will assist health professionals by making recommendations for further investigations based upon physical landmarks and a composite based on parental landmarks when available. As well, patients in remote or rural areas would have access to this invention through telemedicine since their images can be sent electronically for evaluation, avoiding an often difficult inconvenience of travelling long distances to see a large medical center to see a specialist for an initial screening.

Veterinary use: A breed standard is a term used in animal husbandry or fancy which denotes a set of guidelines that are used to ensure that the animals used by breeders conform to the specifics of a breed, and thus used to define the “ideal” of the breed. These standards can vary between associations in the same country as well as between countries. The invention described herein automates the process of judging to produce a reproducible breed standard applicable and made specific to each association.

### DETAILED DESCRIPTION OF THE PREFERRED EMBODIMENT

The invention has four applications: prenatal scanning; postnatal scanning; dysmorphology/periodic surveillance; and veterinary screening.

For the prenatal (fetal) scanning application, the invention can be a computer program that will automate a process of fetal anatomy scanning for malformations using anthropormorphic recognition-like software algorithms. In existing imaging systems an operator manually marks and measures anatomical features, which is highly time-consuming. These measurements and physical characteristics are then used to provide an assessment of the anatomy of a fetus. Many of the measurements are not only difficult and time consuming to obtain, but since they are manually captured they can be highly variable and are open to error since they are operator-dependent. If any abnormalities are detected upon imaging, invasive techniques are often employed, such as amniocentesis or chorionic villi sampling, that can put the viability of a fetus at risk.

By automating the process this invention will decrease the risk of harm to the mother and fetus and give a more accurate risk assessment of congenital or developmental malformations of the child in a noninvasive manner. Automating the process also increases the ability to offer this screening in areas where there is a lack of skilled operators. Measurements, such as the crown to rump length, which is the length of a human from the top of the head to the buttocks, and/or the measurements of long bones such as the femur and humerus, will be acquired and compared to a database of measurements appropriate for the fetal age. These measurements will then be compared with existing measurements for many genetic and developmental syndromes using a computer program. Simultaneously, a comparison will be made between an “idealized” composite based upon the reported ethnicity of the parents. This will allow for detection of intrafamilial variance and further help in the diagnosis and treatment of the fetus.

Use of these non-invasive findings can help a couple decide whether to pursue genetic testing and counselling.

For the postnatal (after birth, i.e. children and adults) scanning application, the invention can be a computer program that will automate a process of anatomy assessment scanning for malformations using anthropomorphic recognition-like software algorithms using non-standardized images of individuals and machine learning techniques.

The invention comprises a computer software program that will automate the system of diagnosis based on features that are associated with genetic syndromes and/or congenital and/or developmental abnormalities and a database comprised of thousands or images of affected patients and nonaffected. This is akin to the existing newborn screen conducted in almost all developed countries, where a heel stick is used to procure a blood sample from almost every child born in the developed world to screen for dozens of metabolic syndromes.

These morphological genetic, congenital and developmental abnormalities will be captured using imaging modalities such as ultrasound, two and/or three dimensional infrared detectors, including video, two and/or three dimensional image capture, MRI or CAT scans. Anatomical landmarks, such as the distance measured between the center of the pupils of the two eyes, and/or the measurement between the first and second toes, mathematical landmarks and pseudo-landmarks, will be acquired and compared to a database of measurements appropriate for the individual's age and ethnicity. These measurements, such as of the face, ears and hands, will then be compared with existing measurements for many syndromes using a data analysis processing module compared to a database of thousands of images. Simultaneously, a comparison will be made between an “idealized” composite made from the measurements acquired from the patient's parents and siblings, and the measurements from the patient. This will allow for detection of intrafamilial variance and further help in the diagnosis and treatment of the patient.

For dysmorphology surveillance, the invention is a computer program that will use data captured over the course of a child's development to screen for any genetic and/or development of dysmorphic features, such as microcephaly associated with cerebral palsy, and storage disorders, such as Hurler Syndrome.

The algorithm will include a database of previous well defined images as well as the parents and any biological siblings of the subject when available, who will be assessed and provide the basis of comparison to estimate and create a template of normalcy by which to screen the subject. Anatomical landmarks, which is the distance measured between the center of the pupils of the two eyes, and/or the measurement between the first and second toes, mathematical landmarks and pseudo-landmarks, will be acquired and compared to a database of measurements appropriate for the individual's age.

For veterinary applications, such as canine breed standard determination, the invention is a computer program that will automate a process of anatomy assessment used for canine breed standard determination. Anatomical landmarks and markings are scanned used using recognition-like software algorithms using standardized images of individual dog breeds and machine learning techniques. This invention comprises a computer software program that will automate the system of breed standard determination based on features that are associated with the specific breed. The landmarks will be captured using imaging modalities such as ultrasound, two and/or three dimensional infrared detectors, including video, two and/or three dimensional image capture, MRI or CAT scans.

While certain novel features of the present invention have been shown and described, it will be understood that various omissions, substitutions and changes in the forms and details of the device illustrated and in its operation can be made by those skilled in the art without departing from the spirit of the invention.

I claim:

1. A system for the identification and diagnosis of genetic features, genetic syndromes, congenital and developmental abnormalities of a human or animal target, the system comprising:
a first data collection module, capable of obtaining facial anatomical measurements of the target;
a database of data correlating facial anatomical measurements with a diagnostic data set;
a second data collection module, capable of obtaining facial anatomical measurements of biological relatives of the target;
a data filter module, capable of comparing the target's facial anatomical measurements with the facial anatomical measurements of the biological relatives to produce a filtered set of target facial measurements;
a data processing module, capable of comparing the filtered set of target facial anatomical measurements with the database; and
a reporting module, capable of providing a report of the comparison.

2. The system of claim 1, where the diagnostic data set comprises genetic characteristics.

3. The system of claim 1, where the diagnostic data set comprises genetic syndromes.

4. The system of claim 1, where the diagnostic data set comprises congenital syndromes.

5. The system of claim 1, where the diagnostic data set comprises developmental and exposure related syndromes.

6. The system of claim 1, where the diagnostic data set comprises veterinary breed characteristics.

7. The system of claim 1, further comprising:
a second data processing module, capable of comparing the anatomical measurements over time of the target.

8. The system of claim 1, where at least one of the first and second data collection modules comprise at least one of ultrasound, two dimensional infrared detectors, three dimensional infrared detectors, two dimensional photography, three dimensional photography, video, MRI and CAT scans.

9. The system of claim 1, where the data filtering module comprises a computer processor.

10. The system of claim 1, further comprising
a data processing module for acquiring anatomical landmarks, mathematical landmarks and pseudo-landmarks from the target's facial anatomical measurements, and
comparing the acquired landmarks to a database of measurements appropriate for the target's age.
